(19) United States
(12) Patent Application Publication
(10) Pub. No.: US 2015/0196521 A1
(43) Pub. Date: Jul. 16, 2015

(54) PHARMACEUTICAL COMPOSITIONS COMPRISING 15-HEPE AND METHODS OF TREATING ASTHMA AND LUNG DISORDERS USING SAME

(71) Applicant: Dignity Sciences Limited, Dublin (IE)

(72) Inventors: Mehar Manku, Birmingham (GB); John Climax, Dublin (IE); Kevin Duffy, Collegeville, PA (US)

(21) Appl. No.: 14/595,015

(22) Filed: Jan. 12, 2015

Related U.S. Application Data
(60) Provisional application No. 61/926,052, filed on Jan. 10, 2014.

(51) Int. Cl. A61K 31/202; A61K 45/06

(52) U.S. Cl. CPC A61K 31/202; A61K 45/06

(57) ABSTRACT

The disclosure provides compositions comprising 15-HEPE or derivatives thereof used singly or in combination with other active agents for the treatment and/or prevention of lung diseases and disorders such as asthma.

### CROSS-REFERENCE TO RELATED APPLICATION(S)

This application claims the benefit of U.S. Provisional Patent Application No. 61/926,052 entitled “PHARMACEUTICAL COMPOSITIONS COMPRISING 15-OHEPA AND METHODS OF TREATING ASTHMA AND LUNG DISORDERS USING SAME”, which was filed on Jan. 10, 2014, the contents of which are all incorporated by reference herein.

### TECHNICAL FIELD

The disclosure generally relates to compositions comprising 15-HEPE used singly or in combination with other active agents for the treatment and/or prevention of asthma and lung disorders.

### BACKGROUND

Lung diseases and disorders, such as asthma and COPD, affect millions world-wide.

Asthma is a chronic lung disease that inflames and narrows the airways. The narrowing of the airways is a result of muscle tightening as well as inflammation. The narrowing results in less air flowing into the lungs. Asthmatics tend to react strongly to airborne substances, leading to increases in mucus production; staring a chain reaction toward a full blown asthmatic attack. Symptoms include recurring periods of wheezing, chest tightness, shortness of breath, coughing and in extreme cases, can be fatal.

Asthma affects people of all ages, but it most often starts during childhood. According to one study by de Nijs et al. (Eur. Respir. Rev., vol. 22(127), pp. 44-52 (Mar. 1, 2013)), approximately 95% of asthma patients have their first episode before the age of 6 years. Young males are consistently reported to have more prevalent wheeze and asthma than young females. However, at the age of 11 years, the prevalence of asthma in young males (7.7%) and young females (7.4%) is similar and after 16 years of age the disease is more common in females (6.2%) than in males (4.3%).

The predominant risk factors associated with childhood-onset asthma are genetic predisposition, a family history of allergy and asthma, viral respiratory infections, bacterial colonization, allergic sensitization and tobacco exposure.

Large, prospective follow-up studies have shown that early and persistent sensitizations to perennial allergens are a strong risk factor for asthma in childhood. Furthermore, childhood-onset asthma is typically associated with other atopic diseases, including allergic rhinitis and atopic dermatitis. Even at adult age the amounts of total and specific immunoglobulin IgE are higher in childhood-onset asthma than in adult-onset asthma. Thus, atopy and allergy seem to be closely linked to asthma starting in childhood.

Viral infections are another risk factor for developing early childhood asthma. Long-term follow-up studies have shown that wheezy episodes associated with rhinovirus infection are a strong predictor of asthma by the age of 6 years. However, studies have also reported that children with early wheezy symptoms and a predisposition to asthma and atopy are at increased risk of lower respiratory infection. Therefore, the direction of causality of viral infection and the development of asthma is still unclear.

Pre-natal and post-natal smoke exposure has been linked to asthma and other wheezy disorders, particularly in the first years of life. Also, exposure to traffic-related air pollution may cause asthma in children.

Daily inhaled corticosteroids are highly effective in improving asthma control in childhood-onset allergic asthma.

### 1. Adult-Onset Asthma

Adult-onset asthma, or late-onset asthma, is considered when asthma symptoms represent for the first time during adulthood. Several definitions of adult-onset asthma can be found in the literature. The age at diagnosis determining the term late-onset asthma varies from 12 years of age to ≧65 years of age.

In contrast to childhood-onset asthma, less is known about the prevalence and factors associated with adult-onset asthma. Studies have shown that it mainly effects females, has a low remission rate and is less often associated with allergy and atopic diseases. In addition, many patients with adult-onset asthma have a poor prognosis, with a faster decline in lung function and more severe persistent airflow limitation. New-onset adult severe asthmatics have compromised lung function even if they have asthma of short duration, suggesting that significant loss of lung function occurs at or soon after the initial diagnosis. Still, adult-onset asthma is largely under investigated and far from completely understood.

### 2. Epidemiology of Adult-Onset Asthma

During the past 20 years, overall asthma prevalence has increased by 38%, in parallel with a similar increase in asthma-like symptoms and allergic rhinitis. Although prevalence rates have been studied more extensively in children, most studies confirm a real increase in asthma prevalence among adults as well. The estimated adult incidence of asthma from pooled general population studies appears to be 4.6 cases per 1,000 person-years in females and 3.6 in males, and there is a trend towards a higher incidence with age. The 5-year age- and sex-specific incidence of newly diagnosed asthma in adults 65 years of age is estimated to be 103 per 100,000 people, where two thirds of asthma deaths occur in people aged ≧65 years. Only one study investigated the long-term outcome of adult-onset asthma and changes in asthma severity over time. In this study, 95% of subjects still had an active asthma 5 years after diagnosis, and half of them had moderate-to-severe disease. Due to a relatively low remission rate, the prevalence of asthma in older adults (≧65 years of age) has been reported to be as high as 10%, where females predominate the age group of 64-75 years. Still, these studies probably underestimate the true prevalence and incidence of asthma.

Besides atopy and atopic disorders, asthma can be a comorbid condition of other diseases. Asthma is a common comorbid factor in sickle cell disease (SCD). However, the incidence of asthma in SCD is much higher than expected compared to rates in the general population. Whether “asthma” in SCD is purely related to genetic and environmental factors or rather is the consequence of the underlying hemolytic and inflammatory state is a topic of recent debate. Regardless of the etiology, hypoxemia induced by bronchoconstriction and inflammation associated with asthma exacerbations will contribute to a cycle of sickling and subsequent complications of SCD. Recent studies confirm that asthma predisposes to complications of SCD such as pain crises, acute chest syndrome, and stroke, and is associated with increased mortality.

Chronic obstructive pulmonary disease (COPD) is the fourth leading cause of death in the United States, accounting for more than 110,000 deaths per year. COPD encompasses both chronic bronchitis and emphysema and is characterized by airway narrowing, air trapping, alveolar destruction, and excessive airway inflammation and oxidative stress. Cigarette smoke (CS) is the most common cause of COPD. In COPD there are marked increases in both inflammation and oxidative stress, which can be replicated in mice by exposing them to tobacco smoke.

Presently, there exist numerous regimens for the treatment of lung diseases and disorders, including short-acting bronchodilators (i.e. short-acting beta agonists), long-acting bronchodilators (i.e. long-acting beta agonists), phosphodiesterase-4 (“PDE4”) inhibitors, corticosteroids, leukotriene receptor antagonists, expectorants and methylxanthines. However, such drugs are not always effective at reducing the symptoms, slowing the progression, or reversing the course of the clinical manifestation of the condition. Additionally, current regimens are limited in dose strength due to side effects and by inefficient delivery to a subject's lungs.

### SUMMARY

The present disclosure provides compositions comprising fatty acid agents including, for example, 15-HEPE (also referred to as “15-OHEPA”) used singly, in combination and/or in combination with a second active agent for treatment of lung diseases or disorders, such as asthma or COPD.

The present disclosure also provides methods for treating a lung disease or disorder mediated by 15-HEPE in a subject in need thereof comprising administering to subject a pharmaceutical composition comprising a therapeutically effective amount of 15-HEPE or combinations thereof. In some embodiments, the pharmaceutical composition comprises about 5 μg to about 10,000 mg of 15-HEPE, for example about 50 μg to about 4,000 mg of 15-HEPE. In some embodiments, the method comprises orally administering about 50 mg to about 4,000 mg of 15-HEPE to the subject per day.

The present disclosure also provides methods for treating a lung disease or disorder mediated by 15-HEPE in a subject in need thereof comprising administering to a subject a pharmaceutical composition comprising a therapeutically effective amount of 15-HEPE. In some embodiments, the pharmaceutical composition comprises about 5 μg to about 10,000 mg of 15-HEPE, for example about 50 μg to about 4,000 mg of 15-HEPE. In some embodiments, the method comprises administering about 50 mg to about 4,000 mg of 15-HEPE to the subject per day.

In some embodiments, the pharmaceutical composition comprises one or more pharmaceutically acceptable excipients.

In some embodiments, the pharmaceutical composition comprises a second active agent. In some embodiments, the second active agent is one or more of: short-acting bronchodilators (i.e. short-acting beta agonists), long-acting bronchodilators (i.e. long-acting beta agonists), phosphodiesterase-4 (“PDE4”) inhibitors, corticosteroids, leukotriene receptor antagonists, expectorants and methylxanthines.

In some embodiments, the step of administering comprises delivering the pharmaceutical composition to a lung of the subject by dry-powder inhaler. In some embodiments, the pharmaceutical composition is in dry powder form.

In some embodiments, the step of administering comprises delivering the pharmaceutical composition to a lung of the subject by metered dose inhaler. In some embodiments, the pharmaceutical composition is in powder form. In some embodiments, the pharmaceutical composition further comprises a propellant.

In some embodiments, the step of administering comprises delivering the pharmaceutical composition to a lung of the subject by nebulizer. In some embodiments, the pharmaceutical composition is a solution comprising a solvent system. In some embodiments, the solvent system comprises an alcohol and/or a polyol. In some embodiments, the solvent system does not include an alcohol or a polyol. In some embodiments, the pharmaceutical composition is a suspension.

In some embodiments, the step of administering the pharmaceutical composition comprises on oral dosage form. In some embodiments, compositions of the invention are in the form of solid dosage forms. Non-limiting examples of suitable solid dosage forms include tablets (e.g. suspension tablets, bite suspension tablets, rapid dispersion tablets, chewable tablets, melt tablets, effervescent tablets, bilayer tablets, etc), caplets, capsules (e.g. a soft or a hard gelatin capsule filled with solid and/or liquids), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for oral administration.

In some embodiments, the pharmaceutical composition is formulated for intravenous or intramuscular injection by methods known in the art.

In some embodiments, the method comprises co-administering a pharmaceutical composition comprising 15-HEPE with a second active agent. In some embodiments, the second active agent is co-formulated with the pharmaceutical composition. In some embodiments, the second active agent is administered simultaneously, concomitantly, or within 1 to 24 hours of the pharmaceutical composition comprising 15-HEPE.

In one embodiment, the 15-HEPE is in the form of an ester. In another embodiment, the compositions comprise a C₁-C₅ alkyl ester of 15-HEPE. In another embodiment, the compositions comprise a methyl ester, propyl ester, or butyl ester. In another embodiment the 15-HEPE is in the form of a pharmaceutically acceptable salt.

In another embodiment, the 15-HEPE comprises lithium, mono, di- or triglyceride or any other ester or 15-HEPE. In another embodiment, the 15-HEPE comprises salts, including but not limited to sodium, lysine, ornithine, piperazine or meglumine. In yet another embodiment, the 15-HEPE the free acid form of 15-HEPE.

In one embodiment, 15-HEPE present in a composition of the invention comprises at least 90% by weight 15-HEPE (as the term “15-HEPE” is defined and exemplified herein). 15-HEPE compositions can comprise even higher purity compositions, for example at least 95% by weight 15-HEPE or at least 97% by weight 15-HEPE, wherein the 15-HEPE is any form of 15-HEPE as set forth herein.

In another embodiment, 15-HEPE is present in a composition of the invention in an amount of about 5 μg to about 10,000 mg, about 25 mg to about 7500 mg, 50 mg to about 5000 mg, about 75 mg to about 2500 mg, about 100 mg to about 1000 mg, or about 50 μg to about 1000 μg, for example about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, about 500 μg, about 505 μg, about 510 μg, about 515 μg, about 520 μg, about 525 μg, about 530 μg, about 535 μg, about 540 μg, about 545 μg, about 550 μg, about 555 μg, about 560 μg, about 565 μg, about 570 μg, about 575 μg, about 580 μg, about 585 μg, about 590 μg, about 595 μg, about 600 μg, about 605 μg, about 610 μg, about 615 μg, about 620 μg, about 625 μg, about 630 μg, about 635 μg, about 640 μg, about 645 μg, about 650 μg, about 655 μg, about 660 μg, about 665 μg, about 670 μg, about 675 μg, about 680 μg, about 685 μg, about 690 μg, about 695 μg, about 700 μg, about 705 μg, about 710 μg, about 715 μg, about 720 μg, about 725 μg, about 730 μg, about 735 μg, about 740 μg, about 745 μg, about 750 μg, about 755 μg, about 760 μg, about 765 μg, about 770 μg, about 775 μg, about 780 μg, about 785 μg, about 790 μg, about 795 μg, about 800 μg, about 805 μg, about 810 μg, about 815 μg, about 820 μg, about 825 μg, about 830 μg, about 835 μg, about 840 μg, about 845 μg, about 850 μg, about 855 μg, about 860 μg, about 865 μg, about 870 μg, about 875 μg, about 880 μg, about 885 μg, about 890 μg, about 895 μg, about 900 μg, about 905 μg, about 910 μg, about 915 μg, about 920 μg, about 925 μg, about 930 μg, about 935 μg, about 940 μg, about 945 μg, about 950 μg, about 955 μg, about 960 μg, about 965 μg, about 970 μg, about 975 μg, about 980 μg, about 985 μg, about 990 μg, about 995 μg, about 1000 μg, about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In one embodiment, a composition of the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of other omega-3 fatty acids such eicosapentaenoic acid, alpha linolenic acid, docosahexaenoic acid, docosapentaenoic acid or derivatives thereof.

In another embodiment, 15-HEPE represents at least about 60%, at least about 70%, at least about 80%, at least about 90%, at least about 95%, at least about 97%, at least about 98%, at least about 99%, or 100%, by weight, of all fatty acids present in a composition of the invention.

In one embodiment, the present disclosure provides a method for treating a lung disease or disorder comprising administering a pharmaceutical composition comprising 15-HEPE to a subject in need thereof.

These and other embodiments of the invention are described in further detail below.

### BRIEF DESCRIPTION OF THE DRAWINGS

FIG. 1 shows ventilatory pressure (mm Hg) changes following exposition of nebulized histamine doses after seven consecutive days of gavage with vehicle.

FIG. 2 shows ventilatory pressure (mm Hg) changes following exposition of nebulized histamine doses after seven consecutive days of gavage with bambuterol at 0.27 mg/kg/day.

FIG. 3 shows ventilatory pressure (mm Hg) changes following exposition of nebulized histamine doses after seven consecutive days of gavage with 15-HEPE at 50 mg/kg/day.

FIG. 4 shows ventilatory pressure (mm Hg) changes following exposition of nebulized histamine doses after seven consecutive days of gavage with 15-HEPE at 500 mg/kg/day.

FIG. 5 shows ventilatory pressure (mm Hg) changes following exposition of nebulized histamine doses after seven consecutive days of gavage with vehicle, bambuterol (0.27 mg/kg/day), OH-EPA (50 mg/kg/day) and OH-EPA (500 mg/kg/day).

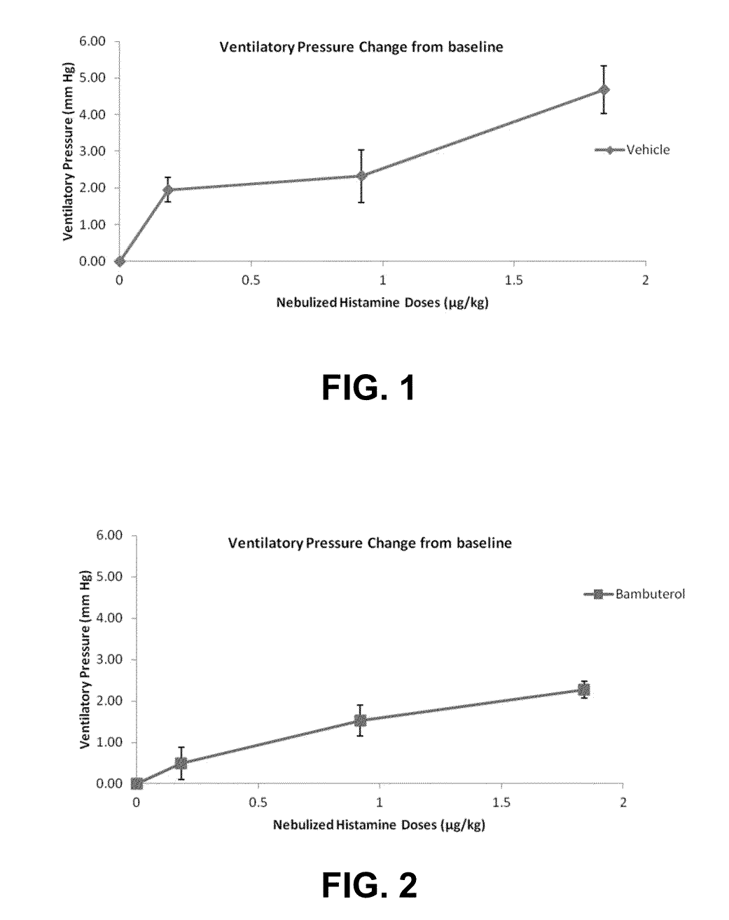

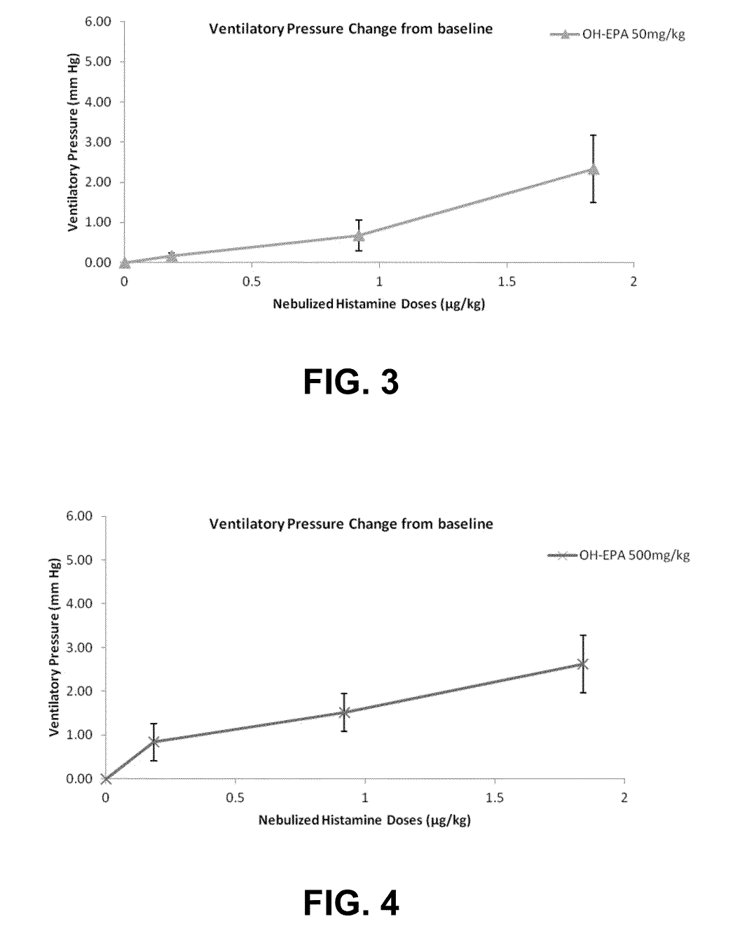

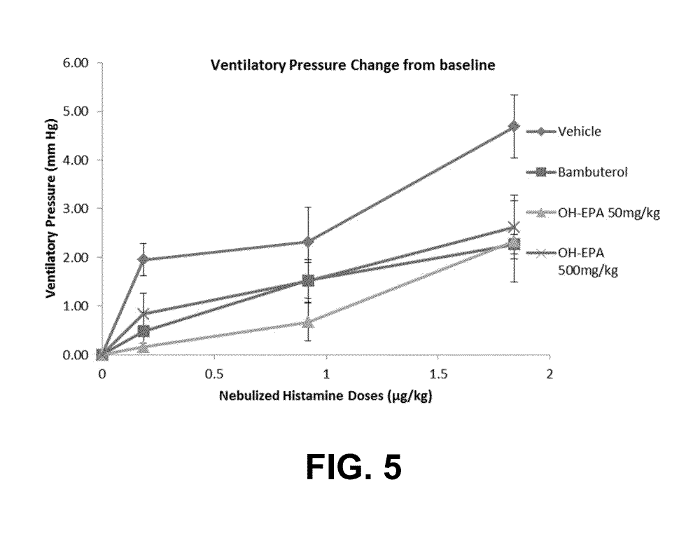

### DETAILED DESCRIPTION

The term “lung disease” as used herein refers to many disorders affecting the lungs, such as asthma, chronic obstructive pulmonary disease (“COPD”), infections like influenza, pneumonia and tuberculosis, lung cancer, and many other breathing problems.

The present disclosure provides compositions (e.g., pharmaceutical compositions) and formulations that comprise 15-HEPE. This agent has been found to improve lung ventilatory pressure in test animals when the animals were challenged with a bronchoconstrictor.

The present disclosure provides compositions comprising 15-HEPE in free acid or derivative form, used singly or in combination with additional active agents including, for example, short-acting bronchodilators (i.e. short-acting beta agonists), long-acting bronchodilators (i.e. long-acting beta agonists), phosphodiesterase-4 (“PDE4”) inhibitors, corticosteroids, leukotriene receptor antagonists, expectorants and methylxanthines.

In some embodiments, the compositions comprise about 50 μg/mL to about 1,000 μg/mL of 15-HEPE or derivative thereof. Contemplated combinations include, without limitation, 15-HEPE and ipratropium, 15-HEPE and albuterol, 15-HEPE and levalbuterol, 15-HEPE with albuterol and levalbuterol, 15-HEPE and tiotropium, 15-HEPE and salmeterol, 15-HEPE and formoterol, 15-HEPE and aformoterol, 15-HEPE and prednisone, 15-HEPE and guaifenesin, 15-HEPE and aminophylline, 15-HEPE and theophylline, 15-HEPE and montelukast, 15-HEPE and zafirlukast, 15-HEPE and zileuton, 15-HEPE and pranlukast. In some embodiments, a composition comprising 15-HEPE includes a therapeutically effective amount of an additional active agent. In some embodiments, a composition comprising 15-HEPE includes less than a therapeutically effective amount of an additional active agent.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

15-Hydroxy-eicosa-5,8,11,13,17-pentaenoic acid (“15-HEPE”) is a derivative of EPA. As used herein, the term “15-HEPE” refers to 15-HEPE in its free acid form (e.g, 15-hydroxy-eicosa-5,8,11,13,17-pentaenoic acid) and/or a pharmaceutically acceptable ester, derivative, conjugate or salt thereof, or mixtures of any of the foregoing. In some embodiments, the 15-HEPE is in the form of a C₁₋₄ alkyl ester such as methyl ester or ethyl ester form.

As used herein, the terms “15-HEPE derivative” and “derivative of 15-HEPE” refer to compounds formed from the chemical conversion of 15-HEPE including, without limitation, esters, derivatives, conjugates or salts thereof, or mixtures of any of the foregoing. One of skill in the art will readily recognize from the chemical structure and other properties whether a given compound is a 15-HEPE derivative.

In various embodiments, the invention provides pharmaceutical compositions, for example aerosolizable compositions, comprising of 15-HEPE.

In one embodiment, the present disclosure provides pharmaceutical compositions comprising, for example, an amount (e.g., a therapeutically effective amount) of 15-HEPE. In one embodiment, the pharmaceutical composition comprises about 50 μg/mL to about 1,000 μg/mL of 15-HEPE, for example 50 μg/mL, 60 μg/mL, 70 μg/mL, 80 μg/mL, 90 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 μg/mL, 260 μg/mL, 270 μg/mL, 280 μg/mL, 290 μg/mL, 300 μg/mL, 310 μg/mL, 320 μg/mL, 330 μg/mL, 340 μg/mL, 350 μg/mL, 360 μg/mL, 370 μg/mL, 380 μg/mL, 390 μg/mL, 400 μg/mL, 410 μg/mL, 420 μg/mL, 430 μg/mL, 440 μg/mL, 450 μg/mL, 460 μg/mL, 470 μg/mL, 480 μg/mL, 490 μg/mL, 500 μg/mL, 510 μg/mL, 520 μg/mL, 530 μg/mL, 540 μg/mL, 550 μg/mL, 560 μg/mL, 570 μg/mL, 580 μg/mL, 590 μg/mL, 600 μg/mL, 610 μg/mL, 620 μg/mL, 630 μg/mL, 640 μg/mL, 650 μg/mL, 660 μg/mL, 670 μg/mL, 680 μg/mL, 690 μg/mL, 700 μg/mL, 710 μg/mL, 720 μg/mL, 730 μg/mL, 740 μg/mL, 750 μg/mL, 760 μg/mL, 770 μg/mL, 780 μg/mL, 790 μg/mL, 800 μg/mL, 810 μg/mL, 820 μg/mL, 830 μg/mL, 840 μg/mL, 850 μg/mL, 860 μg/mL, 870 μg/mL, 880 μg/mL, 890 μg/mL, 900 μg/mL, 910 μg/mL, 920 μg/mL, 930 μg/mL, 940 μg/mL, 950 μg/mL, 960 μg/mL, 970 μg/mL, 980 μg/mL, 990 μg/mL, or 1,000 μg/mL of 15-HEPE.

In one embodiment, the pharmaceutical composition further comprises an additional active agent. In one embodiment, the pharmaceutical composition comprises an amount of the additional active agent that is less than the generally recognized therapeutically effective amount for that agent. In one embodiment, the pharmaceutical composition comprises an amount of the additional active agent that is equal to or greater than the generally recognized therapeutically effective amount for that agent. In one embodiment, the additional active agent has not previously been recognized as effective in the treatment of, including inhibition of, symptoms associated with lung diseases or disorders. In another embodiment, the additional active agent is approved for use in the treatment or prevention of a lung disease or disorder and/or the reduction of, including inhibition of, symptoms associated with a lung disease or disorder.

In one embodiment, the additional active agent is a short-acting bronchodilator. As used herein, the term “short-acting bronchodilator” refers to the class of compounds that are generally recommended for treatment of intermittent symptoms associated with a lung disease or disorder. For example and without limitation, short-acting bronchodilators include anticholinergics (e.g., ipratropium), beta-2-agonists (e.g., albuterol, levalbuterol), and combinations thereof (e.g., albuterol and ipratropium). In some embodiments, the method comprises administering the pharmaceutical composition comprising 15-HEPE and a short-acting bronchodilator in an amount sufficient to provide about 5 μg to about 2,000 μg of short-acting bronchodilator to the subject per day, for example 5 μg per day, 10 μg per day, 18 μg per day, 20 μg per day, 25 μg per day, 30 μg per day, 35 μg per day, 36 μg per day, 40 μg per day, 45 μg per day, 50 μg per day, 55 μg per day, 60 μg per day, 65 μg per day, 70 μg per day, 75 μg per day, 80 μg per day, 85 μg per day, 90 μg per day, 95 μg per day, 100 μg per day, 105 μg per day, 110 μg per day, 115 μg per day, 120 μg per day, 125 μg per day, 130 μg per day, 135 μg per day, 140 μg per day, 145 μg per day, 150 μg per day, 155 μg per day, 160 μg per day, 165 μg per day, 170 μg per day, 175 μg per day, 180 μg per day, 185 μg per day, 190 μg per day, 195 μg per day, 200 μg per day, 205 μg per day, 210 μg per day, 215 μg per day, 220 μg per day, 225 μg per day, 230 μg per day, 235 μg per day, 240 μg per day, 245 μg per day, 250 μg per day, 255 μg per day, 260 μg per day, 265 μg per day, 270 μg per day, 275 μg per day, 280 μg per day, 285 μg per day, 290 μg per day, 295 μg per day, 300 μg per day, 305 μg per day, 310 μg per day, 315 μg per day, 320 μg per day, 325 μg per day, 330 μg per day, 335 μg per day, 340 μg per day, 345 μg per day, 350 μg per day, 355 μg per day, 360 μg per day, 365 μg per day, 370 μg per day, 375 μg per day, 380 μg per day, 385 μg per day, 390 μg per day, 395 μg per day, 400 μg per day, 405 μg per day, 410 μg per day, 415 μg per day, 420 μg per day, 425 μg per day, 430 μg per day, 435 μg per day, 440 μg per day, 445 μg per day, 450 μg per day, 455 μg per day, 460 μg per day, 465 μg per day, 470 μg per day, 475 μg per day, 480 μg per day, 485 μg per day, 490 μg per day, 495 μg per day, 500 μg per day, 525 μg per day, 550 μg per day, 575 μg per day, 600 μg per day, 625 μg per day, 650 μg per day, 675 μg per day, 700 μg per day, 725 μg per day, 750 μg per day, 775 μg per day, 800 μg per day, 825 μg per day, 850 μg per day, 875 μg per day, 900 μg per day, 925 μg per day, 950 μg per day, 975 μg per day, 1,000 μg per day, 1,025 μg per day, 1,050 μg per day, 1,075 μg per day, 1,100 μg per day, 1,125 μg per day, 1,150 μg per day, 1,175 μg per day, 1,200 μg per day, 1,225 μg per day, 1,250 μg per day, 1,275 μg per day, 1,300 μg per day, 1,325 μg per day, 1,350 μg per day, 1,375 μg per day, 1,400 μg per day, 1,425 μg per day, 1,450 μg per day, 1,475 μg per day, 1,500 μg per day, 1,525 μg per day, 1,550 μg per day, 1,575 μg per day, 1,600 μg per day, 1,625 μg per day, 1,650 μg per day, 1,675 μg per day, 1,700 μg per day, 1,725 μg per day, 1,750 μg per day, 1,775 μg per day, 1,800 μg per day, 1,825 μg per day, 1,850 μg per day, 1,875 μg per day, 1,900 μg per day, 1,925 μg per day, 1,950 μg per day, 1,975 μg per day, or 2,000 μg per day.

In one embodiment, the additional active agent is a long-acting bronchodilator. As used herein, the term “long-acting bronchodilator” refers to the class of compounds that are generally recommended for treatment of persistent symptoms associated with a lung disease or disorder. For example and without limitation, long-acting bronchodilators include anticholinergics (e.g., tiotropium), beta-2-agonists (e.g., salmeterol, formoterol, arformoterol), and combinations thereof. In some embodiments, the method comprises administering the pharmaceutical composition comprising 15-HEPE and a long-acting bronchodilator in an amount sufficient to provide about 5 μg to about 100 μg of long-acting bronchodilator to the subject per day, for example 5 μg per day, 6 μg per day, 7 μg per day, 8 μg per day, 9 μg per day, 10 μg per day, 11 μg per day, 12 μg per day, 13 μg per day, 14 μg per day, 15 μg per day, 16 μg per day, 17 μg per day, 18 μg per day, 19 μg per day, 20 μg per day, 21 μg per day, 22 μg per day, 23 μg per day, 24 μg per day, 25 μg per day, 26 μg per day, 27 μg per day, 28 μg per day, 29 μg per day, 30 μg per day, 31 μg per day, 32 μg per day, 33 μg per day, 34 μg per day, 35 μg per day, 36 μg per day, 37 μg per day, 38 μg per day, 39 μg per day, 40 μg per day, 41 μg per day, 42 μg per day, 43 μg per day, 44 μg per day, 45 μg per day, 46 μg per day, 47 μg per day, 48 μg per day, 49 μg per day, 50 μg per day, 51 μg per day, 52 μg per day, 53 μg per day, 54 μg per day, 55 μg per day, 56 μg per day, 57 μg per day, 58 μg per day, 59 μg per day, 60 μg per day, 61 μg per day, 62 μg per day, 63 μg per day, 64 μg per day, 65 μg per day, 66 μg per day, 67 μg per day, 68 μg per day, 69 μg per day, 70 μg per day, 71 μg per day, 72 μg per day, 73 μg per day, 74 μg per day, 75 μg per day, 76 μg per day, 77 μg per day, 78 μg per day, 79 μg per day, 80 μg per day, 81 μg per day, 82 μg per day, 83 μg per day, 84 μg per day, 85 μg per day, 86 μg per day, 87 μg per day, 88 μg per day, 89 μg per day, 90 μg per day, 91 μg per day, 92 μg per day, 93 μg per day, 94 μg per day, 95 μg per day, 96 μg per day, 97 μg per day, 98 μg per day, 99 μg per day, or 100 μg per day.

In one embodiment, the additional active agent is a phosphodiesterase-4 (“PDE4”) inhibitor. As used herein, the terms “phosphodiesterase-4 inhibitor” and “PDE4” inhibitor each refer to the class of compounds that are generally recommended to help prevent exacerbations associated with COPD (e.g., roflumilast). In some embodiments, the method comprises administering the pharmaceutical composition comprising 15-HEPE and a PDE4 inhibitor in an amount sufficient to provide about 250 μg to about 1,000 μg of PDE4 inhibitor to the subject per day, for example 250 μg per day, 275 μg per day, 300 μg per day, 325 μg per day, 350 μg per day, 375 μg per day, 400 μg per day, 425 μg per day, 450 μg per day, 475 μg per day, 500 μg per day, 525 μg per day, 550 μg per day, 575 μg per day, 600 μg per day, 625 μg per day, 650 μg per day, 675 μg per day, 700 μg per day, 725 μg per day, 750 μg per day, 775 μg per day, 800 μg per day, 825 μg per day, 850 μg per day, 875 μg per day, 900 μg per day, 925 μg per day, 950 μg per day, 975 μg per day, or 1,000 μg per day. In one embodiment, the method comprises separately co-administering a PDE4 inhibitor and a pharmaceutical composition comprising 15-HEPE.

In one embodiment, the additional active agent is a corticosteroid. As used herein, the term “corticosteroid” refers to the class of compounds that are generally recommended to treat asthma, COPD exacerbation, stable symptoms of COPD, or progressing symptoms of COPD. For example and without limitation, corticosteroids include methylprednisolone, prednisolone, prednisone, beclomethasone, budesonide, flunisolide, fluticasone, mometasone, triamcinolone and combinations thereof. In some embodiments, the method comprises administering the pharmaceutical composition comprising 15-HEPE and a corticosteroid in an amount sufficient to provide about 50% to about 200% of the daily recommended corticosteroid dose to the subject per day, for example 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% of the daily recommended corticosteroid dose per day. For avoidance of doubt, and not limiting in any way, the amount of a corticosteroid having a daily recommended dose of 40 mg per day to 80 mg per day could be present in a pharmaceutical formulation as disclosed herein (or administered according to a method disclosed herein) in an amount sufficient to provide from about 20 mg of the corticosteroid per day (i.e., 50% of the lowest recommended daily dose, 40 mg/day) to about 160 mg of the corticosteroid per day (i.e., 200% of the maximum daily recommended dose, 80 mg/day). In one embodiment, the corticosteroid is coformulated with the pharmaceutical composition comprising 15-HEPE. In one embodiment, the corticosteroid is in tablet or capsule form. In one embodiment, the method comprises separately co-administering a corticosteroid and a pharmaceutical composition comprising 15-HEPE.

In one embodiment, the additional active agent is an expectorant. As used herein, the term “expectorant” refers to the class of compounds that are generally recommended to help prevent mucus from sticking to the airways of the lungs (e.g., guaifenesin). In some embodiments, the method comprises administering the pharmaceutical composition comprising 15-HEPE and an expectorant in an amount sufficient to provide about 100 mg to about 3,000 mg of expectorant to the subject per day, for example 100 mg per day, 200 mg per day, 300 mg per day, 400 mg per day, 500 mg per day, 600 mg per day, 700 mg per day, 800 mg per day, 900 mg per day, 1,000 mg per day, 1,100 mg per day, 1,200 mg per day, 1,300 mg per day, 1,400 mg per day, 1,500 mg per day, 1,600 mg per day, 1,700 mg per day, 1,800 mg per day, 1,900 mg per day, 2,000 mg per day, 2,100 mg per day, 2,200 mg per day, 2,300 mg per day, 2,400 mg per day, 2,500 mg per day, 2,600 mg per day, 2,700 mg per day, 2,800 mg per day, 2,900 mg per day, or 3,000 mg per day. In one embodiment, the method comprises separately co-administering an expectorant and a pharmaceutical composition comprising 15-HEPE.

In one embodiment, the additional active agent is a methylxanthine. As used herein, the term “methylxanthine” refers to the class of compounds that are generally recommended for relaxing the airways in the lungs, increasing the strength of the diaphragm, stimulating the breathing control centers in the brain, and/or remove mucus from the lungs. For example and without limitation, methylxanthines include aminophylline, theophylline and combinations thereof. In some embodiments, the method comprises administering the pharmaceutical composition comprising 15-HEPE and a methylxanthine in an amount sufficient to provide about 100 mg to about 3,000 mg of methylxanthine to the subject per day, for example 100 mg per day, 200 mg per day, 300 mg per day, 400 mg per day, 500 mg per day, 600 mg per day, 700 mg per day, 800 mg per day, 900 mg per day, 1,000 mg per day, 1,100 mg per day, 1,200 mg per day, 1,300 mg per day, 1,400 mg per day, 1,500 mg per day, 1,600 mg per day, 1,700 mg per day, 1,800 mg per day, 1,900 mg per day, 2,000 mg per day, 2,100 mg per day, 2,200 mg per day, 2,300 mg per day, 2,400 mg per day, 2,500 mg per day, 2,600 mg per day, 2,700 mg per day, 2,800 mg per day, 2,900 mg per day, or 3,000 mg per day. In one embodiment, the method comprises separately co-administering an expectorant and a pharmaceutical composition comprising 15-HEPE. In some embodiments, the methylxanthine is administered separately as an intravenous solution. In some embodiments, a loading dose of the methylxanthine is also administered, with or without coadminstration of the 15-HEPE component.

Any pharmaceutically acceptable excipient known to those of skill in the art may be used in pharmaceutical compositions according to the present disclosure. Any excipient selected for use in the therapeutic compositions should be pharmaceutically acceptable and appropriate for the form in which the therapeutic composition will be used, e.g., aerosol, dry powder, capsule, tablet, cream, gel, milk, oil, lotion, and the like. Preferably, the excipient has an affinity for lung tissue, is well tolerated, and stable when used in an amount adequate to provide the desired consistency and ease of application. By way of example only, a pharmaceutical composition according to the present disclosure may comprise one or more of: propellants, preservatives, amphiphilic agents, surfactants, coloring agents, flavorants, buffers, antioxidants, stabilizers, fragrances, emollients, emulsifiers, thickeners, texturizers, and the like.

In one embodiment, the pharmaceutical composition comprises about 0.5 wt. % to about 5 wt. % of a surfactant, for example, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.05 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, about 3.05 wt. %, about 3.1 wt. %, about 3.15 wt. %, about 3.2 wt. %, about 3.25 wt. %, about 3.3 wt. %, about 3.35 wt. %, about 3.4 wt. %, about 3.45 wt. %, about 3.5 wt. %, about 3.55 wt. %, about 3.6 wt. %, about 3.65 wt. %, about 3.7 wt. %, about 3.75 wt. %, about 3.8 wt. %, about 3.85 wt. %, about 3.9 wt. %, about 3.95 wt. %, about 4 wt. %, about 4.05 wt. %, about 4.1 wt. %, about 4.15 wt. %, about 4.2 wt. %, about 4.25 wt. %, about 4.3 wt. %, about 4.35 wt. %, about 4.4 wt. %, about 4.45 wt. %, about 4.5 wt. %, about 4.55 wt. %, about 4.6 wt. %, about 4.65 wt. %, about 4.7 wt. %, about 4.75 wt. %, about 4.8 wt. %, about 4.85 wt. %, about 4.9 wt. %, about 4.95 wt. %, about 5 wt. % of the surfactant.

In one embodiment, the pharmaceutical composition comprises about 0.5 wt. % to about 5 wt. % of an emulsifier, for example, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.05 wt. %, about 1.1 wt. %, about 1.15 wt. %, about 1.2 wt. %, about 1.25 wt. %, about 1.3 wt. %, about 1.35 wt. %, about 1.4 wt. %, about 1.45 wt. %, about 1.5 wt. %, about 1.55 wt. %, about 1.6 wt. %, about 1.65 wt. %, about 1.7 wt. %, about 1.75 wt. %, about 1.8 wt. %, about 1.85 wt. %, about 1.9 wt. %, about 1.95 wt. %, about 2 wt. %, about 2.05 wt. %, about 2.1 wt. %, about 2.15 wt. %, about 2.2 wt. %, about 2.25 wt. %, about 2.3 wt. %, about 2.35 wt. %, about 2.4 wt. %, about 2.45 wt. %, about 2.5 wt. %, about 2.55 wt. %, about 2.6 wt. %, about 2.65 wt. %, about 2.7 wt. %, about 2.75 wt. %, about 2.8 wt. %, about 2.85 wt. %, about 2.9 wt. %, about 2.95 wt. %, about 3 wt. %, about 3.05 wt. %, about 3.1 wt. %, about 3.15 wt. %, about 3.2 wt. %, about 3.25 wt. %, about 3.3 wt. %, about 3.35 wt. %, about 3.4 wt. %, about 3.45 wt. %, about 3.5 wt. %, about 3.55 wt. %, about 3.6 wt. %, about 3.65 wt. %, about 3.7 wt. %, about 3.75 wt. %, about 3.8 wt. %, about 3.85 wt. %, about 3.9 wt. %, about 3.95 wt. %, about 4 wt. %, about 4.05 wt. %, about 4.1 wt. %, about 4.15 wt. %, about 4.2 wt. %, about 4.25 wt. %, about 4.3 wt. %, about 4.35 wt. %, about 4.4 wt. %, about 4.45 wt. %, about 4.5 wt. %, about 4.55 wt. %, about 4.6 wt. %, about 4.65 wt. %, about 4.7 wt. %, about 4.75 wt. %, about 4.8 wt. %, about 4.85 wt. %, about 4.9 wt. %, about 4.95 wt. %, about 5 wt. % of the emulsifier.

In one embodiment, the pharmaceutical composition comprises a stabilizer. In one embodiment, the pharmaceutical composition comprises about 0.1 wt. % to about 5 wt. % of a stabilizer, for example about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.01 wt. %, about 1.02 wt. %, about 1.03 wt. %, about 1.04 wt. %, about 1.05 wt. %, about 1.06 wt. %, about 1.07 wt. %, about 1.08 wt. %, about 1.09 wt. %, about 1.1 wt. %, about 1.11 wt. %, about 1.12 wt. %, about 1.13 wt. %, about 1.14 wt. %, about 1.15 wt. %, about 1.16 wt. %, about 1.17 wt. %, about 1.18 wt. %, about 1.19 wt. %, about 1.2 wt. %, about 1.21 wt. %, about 1.22 wt. %, about 1.23 wt. %, about 1.24 wt. %, about 1.25 wt. %, about 1.26 wt. %, about 1.27 wt. %, about 1.28 wt. %, about 1.29 wt. %, about 1.3 wt. %, about 1.31 wt. %, about 1.32 wt. %, about 1.33 wt. %, about 1.34 wt. %, about 1.35 wt. %, about 1.36 wt. %, about 1.37 wt. %, about 1.38 wt. %, about 1.39 wt. %, about 1.4 wt. %, about 1.41 wt. %, about 1.42 wt. %, about 1.43 wt. %, about 1.44 wt. %, about 1.45 wt. %, about 1.46 wt. %, about 1.47 wt. %, about 1.48 wt. %, about 1.49 wt. %, about 1.5 wt. %, about 1.51 wt. %, about 1.52 wt. %, about 1.53 wt. %, about 1.54 wt. %, about 1.55 wt. %, about 1.56 wt. %, about 1.57 wt. %, about 1.58 wt. %, about 1.59 wt. %, about 1.6 wt. %, about 1.61 wt. %, about 1.62 wt. %, about 1.63 wt. %, about 1.64 wt. %, about 1.65 wt. %, about 1.66 wt. %, about 1.67 wt. %, about 1.68 wt. %, about 1.69 wt. %, about 1.7 wt. %, about 1.71 wt. %, about 1.72 wt. %, about 1.73 wt. %, about 1.74 wt. %, about 1.75 wt. %, about 1.76 wt. %, about 1.77 wt. %, about 1.78 wt. %, about 1.79 wt. %, about 1.8 wt. %, about 1.81 wt. %, about 1.82 wt. %, about 1.83 wt. %, about 1.84 wt. %, about 1.85 wt. %, about 1.86 wt. %, about 1.87 wt. %, about 1.88 wt. %, about 1.89 wt. %, about 1.9 wt. %, about 1.91 wt. %, about 1.92 wt. %, about 1.93 wt. %, about 1.94 wt. %, about 1.95 wt. %, about 1.96 wt. %, about 1.97 wt. %, about 1.98 wt. %, about 1.99 wt. %, about 2 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt % of the stabilizer.

In one embodiment, the pharmaceutical composition comprises one or more antioxidants. In one embodiment, the pharmaceutical composition comprises about 0.01 wt. % to about 2 wt. % of an antioxidant, for example about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.11 wt. %, about 0.12 wt. %, about 0.13 wt. %, about 0.14 wt. %, about 0.15 wt. %, about 0.16 wt. %, about 0.17 wt. %, about 0.18 wt. %, about 0.19 wt. %, about 0.2 wt. %, about 0.21 wt. %, about 0.22 wt. %, about 0.23 wt. %, about 0.24 wt. %, about 0.25 wt. %, about 0.26 wt. %, about 0.27 wt. %, about 0.28 wt. %, about 0.29 wt. %, about 0.3 wt. %, about 0.31 wt. %, about 0.32 wt. %, about 0.33 wt. %, about 0.34 wt. %, about 0.35 wt. %, about 0.36 wt. %, about 0.37 wt. %, about 0.38 wt. %, about 0.39 wt. %, about 0.4 wt. %, about 0.41 wt. %, about 0.42 wt. %, about 0.43 wt. %, about 0.44 wt. %, about 0.45 wt. %, about 0.46 wt. %, about 0.47 wt. %, about 0.48 wt. %, about 0.49 wt. %, about 0.5 wt. %, about 0.51 wt. %, about 0.52 wt. %, about 0.53 wt. %, about 0.54 wt. %, about 0.55 wt. %, about 0.56 wt. %, about 0.57 wt. %, about 0.58 wt. %, about 0.59 wt. %, about 0.6 wt. %, about 0.61 wt. %, about 0.62 wt. %, about 0.63 wt. %, about 0.64 wt. %, about 0.65 wt. %, about 0.66 wt. %, about 0.67 wt. %, about 0.68 wt. %, about 0.69 wt. %, about 0.7 wt. %, about 0.71 wt. %, about 0.72 wt. %, about 0.73 wt. %, about 0.74 wt. %, about 0.75 wt. %, about 0.76 wt. %, about 0.77 wt. %, about 0.78 wt. %, about 0.79 wt. %, about 0.8 wt. %, about 0.81 wt. %, about 0.82 wt. %, about 0.83 wt. %, about 0.84 wt. %, about 0.85 wt. %, about 0.86 wt. %, about 0.87 wt. %, about 0.88 wt. %, about 0.89 wt. %, about 0.9 wt. %, about 0.91 wt. %, about 0.92 wt. %, about 0.93 wt. %, about 0.94 wt. %, about 0.95 wt. %, about 0.96 wt. %, about 0.97 wt. %, about 0.98 wt. %, about 0.99 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, or about 2 wt. % of the one or more antioxidant.

In one embodiment, the pharmaceutical composition comprises a preservative. In one embodiment, the pharmaceutical composition comprises about 0.1 wt. % to about 5 wt. % of a preservative, for example about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, or about 5 wt. % of a preservative.

In one embodiment, the pharmaceutical composition comprises: about 50 μg/mL to about 4,000 mg/mL of one or more of 15-HEPE and optionally a second active agent.

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 8, 1 to 4 or 1 to 2) of times per day, or as many times as needed to elicit a therapeutic response.

In one embodiment, a composition including, for example, a pharmaceutical composition, as disclosed herein is formulated as an aerosol, a capsule, a tablet or other oral dosage form.

Such formulations may be stable and comprise an amount (e.g., a therapeutically effective amount) of 15-HEPE, in combination with one or more second active agents selected from the group consisting of: short-acting bronchodilators (i.e. short-acting beta agonists), long-acting bronchodilators (i.e. long-acting beta agonists), phosphodiesterase-4 (“PDE4”) inhibitors, corticosteroids, leukotriene receptor antagonists, expectorants and methylxanthines.

### 1. Pharmacokinetics/Pharmacodynamics

The pharmacokinetics and/or pharmacodynamics of the compositions comprising 15-HEPE as disclosed herein may be determined by any method known in the art.

In an embodiment, the pharmacodynamics of a composition comprising 15-HEPE as disclosed herein may be examined using a model of a lung disease or disorder. In a more detailed embodiment, the effects of a composition comprising a vehicle, a positive control or 15-HEPE on ventilator pressure in histamine challenged guinea pigs serves as a model for the pharmacodynamics in asthma. In an exemplary method, guinea pigs are first dosed with compositions comprising a vehicle, a positive control or 15-HEPE for seven days. The treated guinea pigs can then be anesthetized and exposed to a nebulized bronchoconstictor to determine if 15-HEPE can reduce the increase in ventilatory pressure induced by a bronchoconstrictor. This serves as an effective model for asthma and other lung diseases.

2. Methods of Treatment of Diseases and/or Disorders

The compositions and formulations disclosed herein may be used in the treatment of diseases and/or disorders including, for example, lung diseases and/or disorders such as asthma or COPD.

In some embodiments, compositions of the invention are in the form of solid dosage forms for oral delivery. Non-limiting examples of suitable solid dosage forms include tablets (e.g. suspension tablets, bite suspension tablets, rapid dispersion tablets, chewable tablets, melt tablets, effervescent tablets, bilayer tablets, etc), caplets, capsules (e.g. a soft or a hard gelatin capsule filled with solid and/or liquids), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for oral administration.

In another embodiment, 15-HEPE is present in a composition of the invention in an amount of about 5 μg to about 10,000 mg, about 25 mg to about 7500 mg, 50 mg to about 5000 mg, about 75 mg to about 2500 mg, about 100 mg to about 1000 mg, or about 50 μg to about 1000 μg, for example about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, about 100 μg, about 105 μg, about 110 μg, about 115 μg, about 120 μg, about 125 μg, about 130 μg, about 135 μg, about 140 μg, about 145 μg, about 150 μg, about 155 μg, about 160 μg, about 165 μg, about 170 μg, about 175 μg, about 180 μg, about 185 μg, about 190 μg, about 195 μg, about 200 μg, about 205 μg, about 210 μg, about 215 μg, about 220 μg, about 225 μg, about 230 μg, about 235 μg, about 240 μg, about 245 μg, about 250 μg, about 255 μg, about 260 μg, about 265 μg, about 270 μg, about 275 μg, about 280 μg, about 285 μg, about 290 μg, about 295 μg, about 300 μg, about 305 μg, about 310 μg, about 315 μg, about 320 μg, about 325 μg, about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, about 500 μg, about 505 μg, about 510 μg, about 515 μg, about 520 μg, about 525 μg, about 530 μg, about 535 μg, about 540 μg, about 545 μg, about 550 μg, about 555 μg, about 560 μg, about 565 μg, about 570 μg, about 575 μg, about 580 μg, about 585 μg, about 590 μg, about 595 μg, about 600 μg, about 605 μg, about 610 μg, about 615 μg, about 620 μg, about 625 μg, about 630 μg, about 635 μg, about 640 μg, about 645 μg, about 650 μg, about 655 μg, about 660 μg, about 665 μg, about 670 μg, about 675 μg, about 680 μg, about 685 μg, about 690 μg, about 695 μg, about 700 μg, about 705 μg, about 710 μg, about 715 μg, about 720 μg, about 725 μg, about 730 μg, about 735 μg, about 740 μg, about 745 μg, about 750 μg, about 755 μg, about 760 μg, about 765 μg, about 770 μg, about 775 μg, about 780 μg, about 785 μg, about 790 μg, about 795 μg, about 800 μg, about 805 μg, about 810 μg, about 815 μg, about 820 μg, about 825 μg, about 830 μg, about 835 μg, about 840 μg, about 845 μg, about 850 μg, about 855 μg, about 860 μg, about 865 μg, about 870 μg, about 875 μg, about 880 μg, about 885 μg, about 890 μg, about 895 μg, about 900 μg, about 905 μg, about 910 μg, about 915 μg, about 920 μg, about 925 μg, about 930 μg, about 935 μg, about 940 μg, about 945 μg, about 950 μg, about 955 μg, about 960 μg, about 965 μg, about 970 μg, about 975 μg, about 980 μg, about 985 μg, about 990 μg, about 995 μg, about 1000 μg, about 5 mg, about 10 mg, about 15 mg, about 20 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In one embodiment, a composition of the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of other omega-3 fatty acids such eicosapentaenoic acid, alpha linolenic acid, docosahexaenoic acid, docosapentaenoic acid or derivatives thereof.

In another embodiment, 15-HEPE represents at least about 60%, at least about 70%, at least about 80%, at least about 90%, at least about 95%, at least about 97%, at least about 98%, at least about 99%, or 100%, by weight, of all fatty acids present in a composition of the invention.

In some embodiments, the present disclosure provides a method for treating a disease or disorder mediated by 15-HEPE comprising administering a pharmaceutical composition comprising 15-HEPE to a subject in need thereof. In some embodiments, the pharmaceutical composition comprises about 50 μg/mL to about 1,000 μg/mL of 15-HEPE, for example about 50 μg/mL, about 60 μg/mL, about 70 μg/mL, about 80 μg/mL, about 90 μg/mL, about 100 μg/mL, about 110 μg/mL, about 120 μg/mL, about 130 μg/mL, about 140 μg/mL, about 150 μg/mL, about 160 μg/mL, about 170 μg/mL, about 180 μg/mL, about 190 μg/mL, about 200 μg/mL, about 210 μg/mL, about 220 μg/mL, about 230 μg/mL, about 240 μg/mL, about 250 μg/mL, about 260 μg/mL, about 270 μg/mL, about 280 μg/mL, about 290 μg/mL, about 300 μg/mL, about 310 μg/mL, about 320 μg/mL, about 330 μg/mL, about 340 μg/mL, about 350 μg/mL, about 360 μg/mL, about 370 μg/mL, about 380 μg/mL, about 390 μg/mL, about 400 μg/mL, about 410 μg/mL, about 420 μg/mL, about 430 μg/mL, about 440 μg/mL, about 450 μg/mL, about 460 μg/mL, about 470 μg/mL, about 480 μg/mL, about 490 μg/mL, about 500 μg/mL, about 510 μg/mL, about 520 μg/mL, about 530 μg/mL, about 540 μg/mL, about 550 μg/mL, about 560 μg/mL, about 570 μg/mL, about 580 μg/mL, about 590 μg/mL, about 600 μg/mL, about 610 μg/mL, about 620 μg/mL, about 630 μg/mL, about 640 μg/mL, about 650 μg/mL, about 660 μg/mL, about 670 μg/mL, about 680 μg/mL, about 690 μg/mL, about 700 μg/mL, about 710 μg/mL, about 720 μg/mL, about 730 μg/mL, about 740 μg/mL, about 750 μg/mL, about 760 μg/mL, about 770 μg/mL, about 780 μg/mL, about 790 μg/mL, about 800 μg/mL, about 810 μg/mL, about 820 μg/mL, about 830 μg/mL, about 840 μg/mL, about 850 μg/mL, about 860 μg/mL, about 870 μg/mL, about 880 μg/mL, about 890 μg/mL, about 900 μg/mL, about 910 μg/mL, about 920 μg/mL, about 930 μg/mL, about 940 μg/mL, about 950 μg/mL, about 960 μg/mL, about 970 μg/mL, about 980 μg/mL, about 990 μg/mL, or about 1000 μg/mL. In some embodiments, the subject is administered about 5 μg to about 4,000 mg of 15-HEPE per day, for example about 5 μg/day to about 10,000 mg, about 25 mg to about 7500 mg, 50 mg to about 5000 mg, about 75 mg to about 2500 mg, about 100 mg to about 1000 mg, or about 50 μg/day to about 1000 μg/day, for example about 5 μg/day, about 10 μg/day, about 15 μg/day, about 20 μg/day, about 25 μg/day, about 30 μg/day, about 35 μg/day, about 40 μg/day, about 45 μg/day, about 50 μg/day, about 55 μg/day, about 60 μg/day, about 65 μg/day, about 70 μg/day, about 75 μg/day, about 80 μg/day, about 85 μg/day, about 90 μg/day, about 95 μg/day, about 100 μg/day, about 105 μg/day, about 110 μg/day, about 115 μg/day, about 120 μg/day, about 125 μg/day, about 130 μg/day, about 135 μg/day, about 140 μg/day, about 145 μg/day, about 150 μg/day, about 155 μg/day, about 160 μg/day, about 165 μg/day, about 170 μg/day, about 175 μg/day, about 180 μg/day, about 185 μg/day, about 190 μg/day, about 195 μg/day, about 200 μg/day, about 205 μg/day, about 210 μg/day, about 215 μg/day, about 220 μg/day, about 225 μg/day, about 230 μg/day, about 235 μg/day, about 240 μg/day, about 245 μg/day, about 250 μg/day, about 255 μg/day, about 260 μg/day, about 265 μg/day, about 270 μg/day, about 275 μg/day, about 280 μg/day, about 285 μg/day, about 290 μg/day, about 295 μg/day, about 300 μg/day, about 305 μg/day, about 310 μg/day, about 315 μg/day, about 320 μg/day, about 325 μg/day, about 330 μg/day, about 335 μg/day, about 340 μg/day, about 345 μg/day, about 350 μg/day, about 355 μg/day, about 360 μg/day, about 365 μg/day, about 370 μg/day, about 375 μg/day, about 380 μg/day, about 385 μg/day, about 390 μg/day, about 395 μg/day, about 400 μg/day, about 405 μg/day, about 410 μg/day, about 415 μg/day, about 420 μg/day, about 425 μg/day, about 430 μg/day, about 435 μg/day, about 440 μg/day, about 445 μg/day, about 450 μg/day, about 455 μg/day, about 460 μg/day, about 465 μg/day, about 470 μg/day, about 475 μg/day, about 480 μg/day, about 485 μg/day, about 490 μg/day, about 495 μg/day, about 500 μg/day, about 505 μg/day, about 510 μg/day, about 515 μg/day, about 520 μg/day, about 525 μg/day, about 530 μg/day, about 535 μg/day, about 540 μg/day, about 545 μg/day, about 550 μg/day, about 555 μg/day, about 560 μg/day, about 565 μg/day, about 570 μg/day, about 575 μg/day, about 580 μg/day, about 585 μg/day, about 590 μg/day, about 595 μg/day, about 600 μg/day, about 605 μg/day, about 610 μg/day, about 615 μg/day, about 620 μg/day, about 625 μg/day, about 630 μg/day, about 635 μg/day, about 640 μg/day, about 645 μg/day, about 650 μg/day, about 655 μg/day, about 660 μg/day, about 665 μg/day, about 670 μg/day, about 675 μg/day, about 680 μg/day, about 685 μg/day, about 690 μg/day, about 695 μg/day, about 700 μg/day, about 705 μg/day, about 710 μg/day, about 715 μg/day, about 720 μg/day, about 725 μg/day, about 730 μg/day, about 735 μg/day, about 740 μg/day, about 745 μg/day, about 750 μg/day, about 755 μg/day, about 760 μg/day, about 765 μg/day, about 770 μg/day, about 775 μg/day, about 780 μg/day, about 785 μg/day, about 790 μg/day, about 795 μg/day, about 800 μg/day, about 805 μg/day, about 810 μg/day, about 815 μg/day, about 820 μg/day, about 825 μg/day, about 830 μg/day, about 835 μg/day, about 840 μg/day, about 845 μg/day, about 850 μg/day, about 855 μg/day, about 860 μg/day, about 865 μg/day, about 870 μg/day, about 875 μg/day, about 880 μg/day, about 885 μg/day, about 890 μg/day, about 895 μg/day, about 900 μg/day, about 905 μg/day, about 910 μg/day, about 915 μg/day, about 920 μg/day, about 925 μg/day, about 930 μg/day, about 935 μg/day, about 940 μg/day, about 945 μg/day, about 950 μg/day, about 955 μg/day, about 960 μg/day, about 965 μg/day, about 970 μg/day, about 975 μg/day, about 980 μg/day, about 985 μg/day, about 990 μg/day, about 995 μg/day, about 1000 μg/day, about 5 mg/day, about 10 mg/day, about 15 mg/day, about 20 mg/day, about 25 mg/day, about 50 mg/day, about 75 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, about 500 mg/day, about 525 mg/day, about 550 mg/day, about 575 mg/day, about 600 mg/day, about 625 mg/day, about 650 mg/day, about 675 mg/day, about 700 mg/day, about 725 mg/day, about 750 mg/day, about 775 mg/day, about 800 mg/day, about 825 mg/day, about 850 mg/day, about 875 mg/day, about 900 mg/day, about 925 mg/day, about 950 mg/day, about 975 mg/day, about 1000 mg/day, about 1025 mg/day, about 1050 mg/day, about 1075 mg/day, about 1100 mg/day, about 1025 mg/day, about 1050 mg/day, about 1075 mg/day, about 1200 mg/day, about 1225 mg/day, about 1250 mg/day, about 1275 mg/day, about 1300 mg/day, about 1325 mg/day, about 1350 mg/day, about 1375 mg/day, about 1400 mg/day, about 1425 mg/day, about 1450 mg/day, about 1475 mg/day, about 1500 mg/day, about 1525 mg/day, about 1550 mg/day, about 1575 mg/day, about 1600 mg/day, about 1625 mg/day, about 1650 mg/day, about 1675 mg/day, about 1700 mg/day, about 1725 mg/day, about 1750 mg/day, about 1775 mg/day, about 1800 mg/day, about 1825 mg/day, about 1850 mg/day, about 1875 mg/day, about 1900 mg/day, about 1925 mg/day, about 1950 mg/day, about 1975 mg/day, about 2000 mg/day, about 2025 mg/day, about 2050 mg/day, about 2075 mg/day, about 2100 mg/day, about 2125 mg/day, about 2150 mg/day, about 2175 mg/day, about 2200 mg/day, about 2225 mg/day, about 2250 mg/day, about 2275 mg/day, about 2300 mg/day, about 2325 mg/day, about 2350 mg/day, about 2375 mg/day, about 2400 mg/day, about 2425 mg/day, about 2450 mg/day, about 2475 mg/day, about 2500 mg/day about 2525 mg/day, about 2550 mg/day, about 2575 mg/day, about 2600 mg/day, about 2625 mg/day, about 2650 mg/day, about 2675 mg/day, about 2700 mg/day, about 2725 mg/day, about 2750 mg/day, about 2775 mg/day, about 2800 mg/day, about 2825 mg/day, about 2850 mg/day, about 2875 mg/day, about 2900 mg/day, about 2925 mg/day, about 2950 mg/day, about 2975 mg/day, about 3000 mg/day, about 3025 mg/day, about 3050 mg/day, about 3075 mg/day, about 3100 mg/day, about 3125 mg/day, about 3150 mg/day, about 3175 mg/day, about 3200 mg/day, about 3225 mg/day, about 3250 mg/day, about 3275 mg/day, about 3300 mg/day, about 3325 mg/day, about 3350 mg/day, about 3375 mg/day, about 3400 mg/day, about 3425 mg/day, about 3450 mg/day, about 3475 mg/day, about 3500 mg/day, about 3525 mg/day, about 3550 mg/day, about 3575 mg/day, about 3600 mg/day, about 3625 mg/day, about 3650 mg/day, about 3675 mg/day, about 3700 mg/day, about 3725 mg/day, about 3750 mg/day, about 3775 mg/day, about 3800 mg/day, about 3825 mg/day, about 3850 mg/day, about 3875 mg/day, about 3900 mg/day, about 3925 mg/day, about 3950 mg/day, about 3975 mg/day, or about 4000 mg/day.

In some embodiments, the pharmaceutical composition is in dry powder form suitable for administration by dry-powder inhaler. In some embodiments, the pharmaceutical composition is in powder form suitable for administration by metered-dose inhaler. In some embodiments, the pharmaceutical composition further comprises a propellant. In some embodiments, the propellant is 1,1,1,2-tetrafluoroethane.

In some embodiments, the pharmaceutical composition is in a form suitable for administration by nebulizer. In some embodiments, the pharmaceutical composition is a solution comprising a solvent system. In some embodiments, the solvent system comprises an alcohol. In some embodiments, the solvent system does not include an alcohol. In some embodiments, the solvent system comprises a polyol. In some embodiments, the solvent system does not include a polyol. In some embodiments, the pharmaceutical composition is a suspension.

In some embodiments, methods of the present disclosure further comprise determining a first forced expiratory volume (“FEV”) value of the subject before administering the pharmaceutical composition; and determining a second FEV value of the subject after administering the pharmaceutical composition. In some embodiments, the second FEV value is greater than or substantially greater than the first FEV value. In some embodiments, the first and second FEV values are the volume of air the subject forcibly exhales in 0.5 seconds, 1.0 second (i.e., “FEV₁”), 2.0 seconds or 3.0 seconds.

In some embodiments, methods of the present disclosure further comprise determining a first FEV₁/FVC (forced vital capacity) value of the subject before administering the pharmaceutical composition, and determining a second FEV₁/FVC value of the subject after administering the pharmaceutical composition. In some embodiments, the second FEV₁/FVC value is greater than or substantially greater than the first FEV₁/FVC value. In some embodiments, the first FEV₁/FVC value is no greater than about 70%. In some embodiments, the second FEV₁/FVC value is greater than about 70%. In some embodiments, the first FEV₁/FVC value is determined after administration of a bronchodilator medication.

In some embodiments, the subject is male and the first FEV₁/FVC value is no greater than about 88% of a predicted FEV₁/FVC value for men having similar age and body composition of said subject. In some embodiments, the subject is female and the first FEV₁/FVC value is no greater than about 89% of a predicted FEV₁/FVC value for women having similar age and body composition of said subject.

In some embodiments, the disease or disorder is selected from the group consisting of COPD or asthma. In some embodiments, the disease or disorder is COPD. In some embodiments, the disease or disorder is asthma.

In some embodiments, methods according to the present disclosure further comprise administering (including co-administering) a second active agent. In some embodiments, the second active agent is selected from the group consisting of: short-acting bronchodilators (i.e. short-acting beta agonists), long-acting bronchodilators (i.e. long-acting beta agonists), phosphodiesterase-4 (“PDE4”) inhibitors, corticosteroids, leukotriene receptor antagonists, expectorants and methylxanthines. In some embodiments, the second active agent is co-formulated with the pharmaceutical composition comprising 15-HEPE. In some embodiments, the second active agent is formulated separately and co-administered with the pharmaceutical composition comprising 15-HEPE. As used herein, the term “co-administer” includes administering the second active agent simultaneously, concomitantly, within 1 hour, within 2 hours, within 3 hours, 4 hours, within 5 hours, within 6 hours, within 7 hours, within 8 hours, within 9 hours, within 10 hours, within 11 hours, within 12 hours, within 13 hours, within 14 hours, within 15 hours, within 16 hours, within 17 hours, within 18 hours, within 19 hours, within 20 hours, within 21 hours, within 22 hours, within 23 hours, or within 24 hours of the pharmaceutical composition comprising 15-HEPE.

In one embodiment, the method comprises administering a pharmaceutical composition as disclosed herein once per day, twice per day, three times per day, or more than three times per day.

In one embodiment, upon treatment in accordance with the present disclosure, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the treated subject exhibits an improvement in FEV value, an improvement in FCV value, or a combination thereof.

As used herein, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, is an amount effective to achieve a desired pharmacologic effect or therapeutic improvement without undue adverse side effects. In other embodiments, it is understood that “an effective amount” or “a therapeutically effective amount” varies from subject to subject, due to variation in metabolism, age, weight, general condition of the subject, the condition being treated, the severity of the condition being treated, and the judgment of the prescribing physician. The term “pharmaceutically acceptable” in the present context means that the substance in question does not produce unacceptable toxicity to the subject or interaction with other components of the composition.

In another embodiment, the present disclosure provides a method of treating, reducing the symptoms of, slowing progression of or promoting regression of a lung disease or disorder such as asthma or COPD.

In one embodiment, the present disclosure provides a method of reducing or preventing side effects associated with administration of a second active agent. Administration of certain second active agents disclosed herein have been associated with various intolerance symptoms, such as but not limited to: irritation of the oropharynx, mouth, mucus tissues, etc.; headache; nausea; upset stomach; severe allergic reactions (rash; hives; difficulty breathing; tightness in the chest; swelling of the mouth, face, lips, or tongue); blood in the urine; bloody vomit; diarrhea; dizziness; excitability; fast breathing; fast/irregular heartbeat; flushing; increased thirst or urination; irritability; muscle twitching; pounding in the chest; restlessness; seizures; stomach pain; trouble sleeping; and vomiting. In one embodiment, a method of reducing side effects associated with administration of a second active agent as disclosed herein comprises discontinuing administration of a first pharmaceutical composition comprising the second active agent and administering to a subject a second pharmaceutical composition comprising 15-HEPE as disclosed herein. In one embodiment, the second pharmaceutical composition includes an amount of a second active agent that is less than the amount of the same second active agent in the first pharmaceutical composition. In one embodiment, the second pharmaceutical composition includes an amount of the second active agent that is about equal to or equal to the amount of that second active agent in the first pharmaceutical composition. In one embodiment, the second pharmaceutical composition includes an amount of the second active agent that is more than the amount of that second active agent in the first pharmaceutical composition. In one embodiment, the second pharmaceutical composition does not include the second active agent, essentially none of the second active agent, or substantially none of the second active agent.

Without further description, it is believed that one of ordinary skill in the art may, using the preceding description and the following illustrative examples, make and utilize the agents of the present disclosure and practice the claimed methods. The following working examples are provided to facilitate the practice of the present disclosure, and are not to be construed as limiting in any way the remainder of the disclosure.

### EXAMPLES

### Example 1

### In Vivo Investigation of Histamine-Induced Bronchoconstriction Following Chronic Exposure to 15-HEPE in Guinea Pigs

The guinea pig is a reliable animal model in respiratory research due to their respiratory sensitivity and their expression of similar respiratory reactions to those observed in humans. Guinea pigs possess a well-developed bronchial smooth muscle compartment, integral to the purpose of this study.

This study design was based on current International Conference on Harmonization (ICH) Harmonized Tripartite Guidelines [ICH S7A] and generally accepted procedures for the testing of pharmaceutical compounds.

Adult male Hartley guinea pigs weighing approximately 450-500 g were acclimated least 5 days before the day of the beginning of the oral gavages. Animals were assigned to one of four groups (low dose, high dose, vehicle and positive control) and were double-housed per group during acclimation period.

A. Preparation of Test Articles Solutions

The test article was formulated using olive oil as the vehicle. A low and a high dose of 15-HEPE (50 mg/kg and 500 mg/kg) was formulated.

B. Preparation of Positive Control Solution

A 1 mg/ml stock solution of bambuterol was prepared in an aqueous solution, and was then mixed with olive oil in the gavage syringe for the vehicle effect. The stock solution was stored at −20° C., but was used at room temperature and was considered stable for the duration of the experiment.

C. Preparation of the Bronchoconstrictor

A 5 μg/ml stock solution of histamine dihydrochloride (Sigma Aldrich) was prepared in a saline solution (0.9% NaCl). The stock solution was stored at 4° C. The expiration date was set at 14 days after preparation.

D. Study Design

The concentrations of positive control, (bambuterol 0.27 mg/kg/day) and the hypertensor (histamine 0.184, 0.919 and 1.84 μg/kg) were selected based on literature references.

Each group received the designated compound by oral gavage using a 1 ml syringe. The dosing schedule was as follows:
Vehicle (olive oil)
Bambuterol 0.27 mg/kg/day
15-HEPE 50 mg/kg/day
15-HEPE 500 mg/kg/day

The stock solutions were administered once per day for seven consecutive days. On the seventh dosage day the animals underwent surgery two hours after the oral feeding. All animal care and vivarium maintenance were recorded, with documents kept at the test facility.

Guinea pigs were anesthetized with an intraperitoneal injection of urethane (1.5 g/kg). A tracheotomy was performed and the animals were mechanically ventilated throughout the experiment. The ventilatory pressure was recorded and analyzed for signs of bronchospasm. A pulse oxymeter was attached to each animal's hind paw to obtain heart beat and oxygen saturation on the anaesthetized guinea pigs to continuously monitor the general condition of the animals during the surgical procedure. Following baseline measurements, three doses of histamine (0.184, 0.919 and 1.84 μg/kg) were nebulized (constant volume tidal, 60 respirations per minute, 7.5 ml/sec), with an interval of 20 minutes between each dose, or until a steady state was reached. Respiratory resistance, heart rate, and oxygen saturation were monitored continuously for a period of about 1 hour post challenge. At the end of the experiment, the animals were euthanized by exsanguination.

E. Results and Analysis

A networked personal computer running Microsoft Windows was used for data acquisition. The acquisition software was Axoscope 10.2 by Axon Instruments. Axoscope 10.2 has been fully validated in the connected context in which it was used for this study.

Clampfit 10.2.0.14 analysis software (Axon Instruments), installed on networked personal computers running Microsoft Windows was used. Clampfit 10.2.0.14 has been fully validated in the connected context in which it was used. The graphics software for illustrations is Microsoft Office Excel 2007 installed on networked personal computers running Microsoft Windows. The ventilatory pressure (respiratory resistance) was recorded continuously and was used to calculate the mean respiratory resistance. One-way ANOVAs comparing pre- and post-exposure parameters across experimental groups. Statistical significance confirmed at p≦0.05.

TABLE 1
Changes in ventilator pressure (mm Hg) from baseline following nebulized
histamine doses after seven consecutive days of gavage with vehicle.

| | |
|---|---|
| Treatment | Vehicle Change from baseline |

Respiratory Resistance

| Conditions | Guinea pig | Guinea pig | Guinea pig | Average | | | Ttest (vs | |
|---|---|---|---|---|---|---|---|---|
| (μg/kg) | #1 | #2 | #3 | RR | STED | SEM | baseline) | n |
| Baseline | 0.00000 | 0.00000 | 0.00000 | 0.00 | 0.0000 | 0.0000 | N/A | 3 |
| 0.184 | 1.65227 | 1.59524 | 2.61711 | 1.95 | 0.5742 | 0.3315 | 0.0276 | 3 |
| 0.919 | 2.57242 | 0.98181 | 3.41396 | 2.32 | 1.2352 | 0.7131 | 0.0827 | 3 |
| 1.84 | 5.98946 | 3.99966 | 4.08134 | 4.69 | 1.1260 | 0.6501 | 0.0187 | 3 |

TABLE 2
Changes in ventilator pressure (mm Hg) from baseline following nebulized histamine
doses after seven consecutive days of gavage with bambuterol (0.27 mg/kg/day).

| | |
|---|---|
| Treatment | Bambuterol Change from baseline |

Respiratory Resistance

| | Guinea pig | Guinea pig | Guinea pig | Average | | | Ttest (vs | |
|---|---|---|---|---|---|---|---|---|
| Conditions | #1 | #2 | #3 | VP | STED | SEM | vehicle) | n |
| Baseline | 0.00000 | 0.00000 | 0.00000 | 0.00 | 0.0000 | 0.0000 | N/A | 3 |
| 0.184 | 0.27589 | −0.05857 | 1.23766 | 0.48 | 0.6729 | 0.3885 | 0.1337 | 3 |
| 0.919 | 1.09894 | 1.21916 | 2.26570 | 1.53 | 0.6417 | 0.3705 | 0.3786 | 3 |
| 1.84 | 2.21483 | 1.94511 | 2.65102 | 2.27 | 0.3562 | 0.2057 | 0.0238 | 3 |

TABLE 3
Changes in ventilator pressure (mm Hg) from baseline following nebulized histamine
doses after seven consecutive days of gavage with 15-HEPE (50 mg/kg/day).

| | |
|---|---|
| Treatment | OH-EPA 500 mg/kg Change from baseline |

Respiratory Resistance

| | Guinea pig | Guinea pig | Guinea pig | Average | | | Ttest (vs | |
|---|---|---|---|---|---|---|---|---|
| Conditions | #1 | #2 | #3 | VP | STED | SEM | vehicle) | n |
| Baseline | 0.00000 | 0.00000 | 0.00000 | 0.00 | 0.0000 | 0.0000 | N/A | 3 |
| 0.184 | 0.20979 | 1.65689 | 0.64889 | 0.84 | 0.7420 | 0.4284 | 0.2692 | 3 |
| 0.919 | 0.84943 | 1.37021 | 2.33968 | 1.52 | 0.7563 | 0.4366 | 0.3913 | 3 |
| 1.84 | 2.16880 | 1.79098 | 3.91334 | 2.62 | 1.1321 | 0.6536 | 0.0885 | 3 |

TABLE 4
Changes in ventilator pressure (mm Hg) from baseline following nebulized histamine
doses after seven consecutive days of gavage with 15-HEPE (500 mg/kg/day).

| | |
|---|---|
| Treatment | OH-EPA 500 mg/kg Change from baseline |

Respiratory Resistance

| | Guinea pig | Guinea pig | Guinea pig | Average | | | Ttest (vs | |
|---|---|---|---|---|---|---|---|---|
| Conditions | #1 | #2 | #3 | RR | STED | SEM | vehicle) | n |
| Baseline | 0.00000 | 0.00000 | 0.00000 | 0.00 | 0.0000 | 0.0000 | N/A | 3 |
| 0.184 | 0.20979 | 1.65689 | 0.64889 | 0.84 | 0.7420 | 0.4284 | 0.2108 | 3 |
| 0.919 | 0.84943 | 1.37021 | 2.33968 | 1.52 | 0.7563 | 0.4366 | 0.1636 | 3 |
| 1.84 | 2.16880 | 1.79098 | 3.91334 | 2.62 | 1.1321 | 0.6536 | 0.0949 | 3 |

From the foregoing, it will be appreciated that specific embodiments of the invention have been described herein for purposes of illustration, but that various modifications may be made without deviating from the scope of the invention. Accordingly, the invention is not limited except as by the appended claims.

I/We claim:

1. A method for treating a lung disease or disorder comprising administering a pharmaceutical composition comprising 15-HEPE to a subject in need thereof.

2. The method of claim 1, wherein the pharmaceutical composition comprises about 50 μg to about 4,000 mg of 15-HEPE.

3. The method of claim 1, wherein the subject is administered an amount of the composition sufficient to provide about 1 to about 2,000 mg of 15-HEPE to the subject per day.

4. A method for treating a disease or disorder comprising administering a pharmaceutical composition comprising 15-HEPE to a lung of a subject in need thereof.

5. The method of claim 4, wherein the pharmaceutical composition comprises about 50 μg to about 1,000 μg/mL of 15-HEPE.

6. The method of claim 5, wherein the subject is administered an amount of the composition sufficient to provide about 5 μg to about 1,000 μg of 15-HEPE to the subject per day.

7. The method of claim 6, wherein the disease or disorder is selected from the group consisting of: asthma and COPD.

8. The method of claim 7, wherein the pharmaceutical composition further comprises a second active agent.

9. The method of claim 8, wherein the second active agent is selected from the group consisting of: short-acting bronchodilators, long-acting bronchodilators, phosphodiesterase-4 inhibitors, corticosteroids, leukotriene receptor antagonists, expectorants and methylxanthines.
